(19) United States
(12) Patent Application Publication
(10) Pub. No.: US 2010/0010413 A1
(43) Pub. Date: Jan. 14, 2010

(54) Needle for Subcutaneous Port

(76) Inventors: David A. Loiterman, Oak Brook, IL (US); Michael G. Loiterman, Yorkville, IL (US)

Correspondence Address: VEDDER PRICE P.C., 222 N. LASALLE STREET, CHICAGO, IL, 60601, US

(21) Appl. No.: 12/500,436

(22) Filed: Jul. 9, 2009

Related U.S. Application Data
(60) Provisional application No. 61/079,238, filed on Jul. 9, 2008.
(60) Provisional application No. 61/091,044, filed on Aug. 22, 2008.

(51) Int. Cl. A61M 5/00; A61M 1/14

(52) U.S. Cl. 604/6.16; 604/272; 604/266

(57) ABSTRACT

This disclosure relates to a new type of needed for a subcutaneous port or for any use where blood is recycled, and more precisely to a needle with reduced friction openings for easing blood and its elements along a passageway made of a through bore in the body of a needle. The needle includes an oval shape opening for increased mechanical resistance of the needle while allowing a greater passage curvature of the blood cells at the greatest zone of passage. In other embodiments, a plurality of staggered openings is used to reduce the flow through any single opening where damage occurs, the openings can be made in a curved area, or a plurality of smaller openings or a grid made of openings can be used to further reduce the interference of the needle tip and the needle openings on blood.

### CROSS-REFERENCE TO RELATED APPLICATIONS

The present patent application claims priority from and the benefit of U.S. Provisional Patent Application No. 61/079,238, filed Jul. 9, 2008, entitled Needle for Subcutaneous Port, which prior application is hereby incorporated herein by reference, and U.S. Provisional Patent Application No. 61/091,044, filed on Aug. 22, 2008, also entitled Needle for Subcutaneous Port, which is also hereby incorporated herein by reference.

### FIELD OF THE DISCLOSURE

This disclosure relates to a needle for use with a subcutaneous port with a membrane, to minimize damage caused to blood cells as a result of rapid circulation of blood at the tip of the needle, and more particularly, to a needle for a subcutaneous port with openings and edges capable of protecting blood cells via a reduced local velocity, a reduced friction, and a controlled direction of the flow.

### BACKGROUND

During medical interventions, tubes or catheters are used in a wide variety of applications in conjunction with different medical devices. Small, hollow tubes are introduced within a patient's body to remove bodily fluids, circulate them through external equipment, or to provide access to bodily fluids for equipment. These tubes are often equipped with end needles, also called high flow and low resistance needles, that puncture and are passed through a regenerating layer of skin or into a surface to connect with an internal volume where the fluid is found. Even when sharpened hollow tubes cut the skin or the surface, a rip is made in the shape of a small circle around the periphery of the tube. As a result, part of the surface is cut away or damaged. The removed portion can also become a loose particle entering the fluid to be collected. When punctured, skin also requires additional care and attention to heal properly.

In 1952, U.S. Pat. No. 2,717,600 to Huber first described what is now known in the art as the Huber needle. A hollow cylinder is cut in the shape of a pointed knife where the center circular opening is angled as part of the bladed surface. As a result, the Huber needle creates a small, linear incision as it is inserted and does not remove part of the skin into which it is inserted as long as the medium is allowed to deform plastically around the external body of the Huber needle. FIG. 1 illustrates several Huber needles as contemplated by U.S. Pat. No. 2,717,600.

While Huber needles are designed to minimize the residual trace, their heads are not optimized to limit the pressure drop created in a fluid moving in the Huber needle. For example, in the vicinity of the tip, blood is accelerated locally into a narrow tip and enters the needle head around an edged rim before it must change direction and travel alongside the needle stem. A blood cell hitting the edge of the needle may be damaged. Therefore, a medical device, such as a pump, connected ultimately to a Huber needle requires more energy to operate than if no needle is placed at the tip. Using a Huber needle also results in a need to increase the power at the pump, and thus subject the blood to greater pressure gradients and greater exit velocities as it travels through the length of the needle.

Human blood, unlike a pure liquid, is a bodily fluid composed of different types of cells suspended in a liquid called blood plasma. These cells are fragile and can be damaged easily as they travel up a needle, and more precisely as they enter the tip of a needle. Blood plasma is 90% water and 10% dissolved proteins, glucose, mineral ions, hormones, or different soluble gases such as carbon dioxide. These parts constitute 55% of blood fluid. The remainder of human blood is made of red blood cells and different types of white blood cells, such as neutrophil, eosinophil, basophil, lymphocyte, monocyte, and macrophage cells. The red and white blood cells are not rigid entities floating in the plasma but are viscous bodies having a good degree of flexibility. As the distance between adjacent cells in the blood decreases, the blood increases in viscosity. As the plasma changes consistency, the blood viscosity also increases.

When viscosity of a fluid transported in a tube increases, the force needed to move the fluid also increases since these forces must compensate for contact friction with the internal surface of the tube. Such increased force can result in damage to the fluid. The average viscosity of blood at 37° C. is 0.0027 Ns/m². Many factors can change the viscosity of blood over time, factors such as hemodialysis. As the blood is filtered during dialysis, unwanted waste, generally a portion of the liquid in the blood is removed. Accordingly, the remaining portion of the blood is thickened (i.e., the cells grow closer) in the volume. Plasma viscosity and whole blood viscosity rises with hemodialysis with the degree of ultrafiltration (i.e., weight loss). See The Effect of Hemodialysis on Whole Blood, Plasma and Erythrocyte Viscosity by Wink J., Vaziri N D., Barker S., Hyatt J., and Ritchie C., at Int. J. Artif. Organs., September 1988; 11(5):340-2.

If 5% of the volume of a patient's blood is removed during hemodialysis, the Wink research approximates the increase in viscosity of the blood by the same amount, or about 5%. Patients on hemodialysis sit for long periods of time and may be connected to a machine for up to 8 hours. Their blood can be circulated many times through an artificial kidney. As a result, a large fraction of the blood is removed and the blood is often thickened significantly. Accordingly, the damage on the blood cells at the needle increases as the dialysis time increases unless the needle is designed to protect the blood. Multiple passages of blood at a needle tip, even if damage is minimum for each single passage can result in undesired side effects to the patient.

The average size of the erythrocyte disk in a red blood cell is 6 to 8 μm where 1 μm corresponds to 1×10⁻⁶ m or 0.40×10⁻⁴ in. The average size of the different human white blood cells ranges from 7 to 17 μm for lymphocytes and monocytes, respectively. Since about 50% of the volume of blood is made of blood cells, the average distance between adjacent cells can also be taken to be around 7 to 17 μm (for a total cross-section of 34 μm corresponding to the sum of a cell and the surrounding plasma). To better understand the dynamics at the tip of a high flow/low resistance needle, an average needle opening of 1 mm in size with an opening hole of about 0.75 mm in radius, or 750 μm, is about 20 times the size of the cross-section of the cell moving through the opening hole.

The dynamics of a flow of liquid in an opening differs from the dynamics of a flow of particles through the same opening. For example, sand in an hourglass must have a precise maximum ratio over the size of the opening between the upper and lower cavity to flow freely as a semi-liquid. When blood cells are pushed through an opening having a radius of relative importance compared to the size of the cells, these cells can be damaged if the passage is too narrow, if the passage is too rapid or if the change in direction is abrupt. In addition, the reduced section of the needle tip increases locally the velocity of the cells at the opening, thereby increasing the energy available to damage the cells when they come into contact with the edge of the high flow/low resistance needle opening.

If blood is moved too rapidly, moved repetitively past sharp edges, or pressurized in a choked area of the needle tip, damage to the blood can occur, which may lead to a plurality of unwanted medical conditions. In the case of cyclical and repetitive blood circulating conditions, such as the dialysis treatment of blood where the fluid is passed repeated through a filtering machine, different elements of the blood can be progressively damaged with each passage.

U.S. Pat. No. 5,041,098 (“Loiterman et al.”), which is incorporated herein by reference and is a prior art device co-invented by the inventor of the present disclosure, describes a subcutaneous device used in the dialysis process that must be accessed a plurality of times as the patient undergoes repetitive treatments. The Huber needle described above, while adapted to preserve the silicone-based plenum surface shown as element 20 of FIG. 2 taken from Loiterman et al., results in the creation of a needle only capable of drawing blood near the bottom of the blood-filled cavity 14 at an angle from the bottom of the blood flow in the cavity. The Huber needle is unsuited for this use.

In FIG. 2, Loiterman et al. teaches the use of a sharp needle point with a cpointed tip and a lateral circular opening to draw blood at a mid height of the cavity in a perpendicular flow. In FIG. 3, Loiterman et al. shows the proportion of the size of the needle compared to the blood cavity and illustrates how a bent tip can be used to position the end portion of the needle within the cavity 14. What is needed is a new type of needle designed for repetitive use on an internal port for access of an external device to the blood stream that can be inserted and withdrawn without damage and that is capable of promoting undamaged flow of blood after repetitive passages through the needle opening(s) when the blood is circulated and changes consistency during the process of circulation.

### SUMMARY

This disclosure relates to a new type of needle for a subcutaneous port or for any use where blood is recycled, and more precisely to a needle with reduced friction openings for easing blood and its elements along a passageway made of a through bore in the body of a needle. The needle includes an oval shape opening for increased mechanical resistance of the needle while allowing a greater passage curvature of the blood cells at the greatest zone of passage. In other embodiments, a plurality of staggered openings is used to reduce the flow through any single opening where damage occurs, the openings can be made in a curved area, or a plurality of smaller openings or a grid made of openings can be used to further reduce the interference of the needle tip on the blood.

### BRIEF DESCRIPTION OF THE DRAWINGS

Certain embodiments are shown in the drawings. However, it is understood that the present disclosure is not limited to the arrangements and instrumentality shown in the attached drawings.

FIG. 1 is taken from the prior art and illustrates several Huber syringe needles.

FIG. 2 is taken from the prior art and illustrates a port with one type of known needle.

FIG. 3 is taken from the prior art and illustrates the port of FIG. 2 shown three dimensionally with a bent needle.

FIG. 4 is a port from the prior art with a needle having an oval opening according to a first embodiment of the present disclosure.

FIG. 5A is a detailed front view of the needle of FIG. 4.

FIG. 5B is a detailed cut view of the needle of FIG. 5A along cut line 5B-5B.

FIG. 6 is a port from the prior art with a needle with two staggered openings according to another embodiment of the present disclosure.

FIG. 7 is a port from the prior art with a bent needle according to another embodiment of the present disclosure.

FIG. 8 is a port from the prior art with a needle with a grid portion according to another embodiment of the present disclosure.

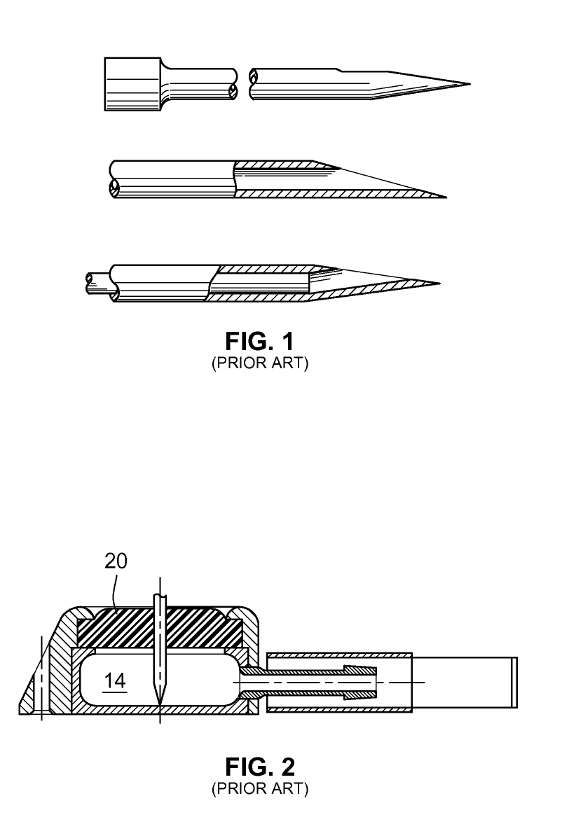

### DETAILED DESCRIPTION OF THE INVENTION

For the purposes of promoting and understanding the principles disclosed herein, reference is now made to the preferred embodiments illustrated in the drawings, and specific language is used to describe the same. It is nevertheless understood that no limitation of the scope of the invention is hereby intended. Such alterations and further modifications in the illustrated devices and such further applications of the principles disclosed and illustrated herein are contemplated as would normally occur to one skilled in the art to which this disclosure relates.

Needles are long, hollow tubes used when placed at one end in a fluid such as a biologic or physiologic fluid to draw the said fluid from the dipped end to the opposite end by applying a pressure differential. Within the scope of this disclosure, the word fluid includes any biologic or physiologic fluid such as, for example, blood or urine. Needles have tips designed to puncture or cut into a solid to reach a destination generally below the surface where the fluid is found. The long axis of the needle contains a hollow tubular channel (or through bore) extending from a proximal end that may be connected to a machine or volume where fluid can be stored. The distal end includes at least one or more orifices. Orifices can be located at various distances along the body of the needle and may be placed in different orientations.

Blood cells are damaged when they travel in the blood and encounter an obstacle. Blood cells can also be damaged if the serum in which they float is placed under a pressure differential that results in the creation of shearing forces within a single blood cell. For example, in a machine a pump can be used to suck blood from a patient. If the needle is connected to a long tube, the pressure at the pump must be sufficient to compensate the pressure drop over the length of the tube. A powerful pump may result locally in damage to the cells.

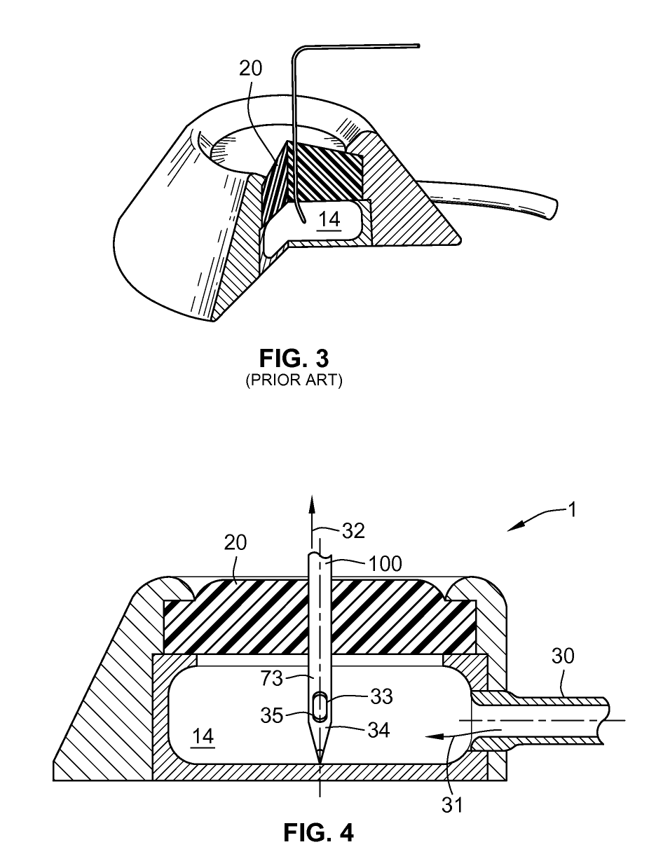

For damage to the blood to be minimized, the pressure drop in the needle tip must be lowered. For example, keeping the blood in a laminar flow while it enters and travels along the length of the needle reduces the pressure drop compared to any turbulent flow of blood. Another method of reducing the pressure loss through the needle is to change the geometrical parameters of the opening or the bore to prevent friction. For example, if the needle's internal surface area is A, and opening area is a fraction of A, the speed of the fluid through the opening will be a multiple of the speed in the needle body. This change in velocity may result in turbulent flow if the Reynolds number of the blood reaches a certain fixed value based on fluid viscosity. In addition, the blood located in the cavity or fluid reservoir 14 must change direction, velocity, and travel upwards through the needle as shown by arrow 32 on FIG. 4.

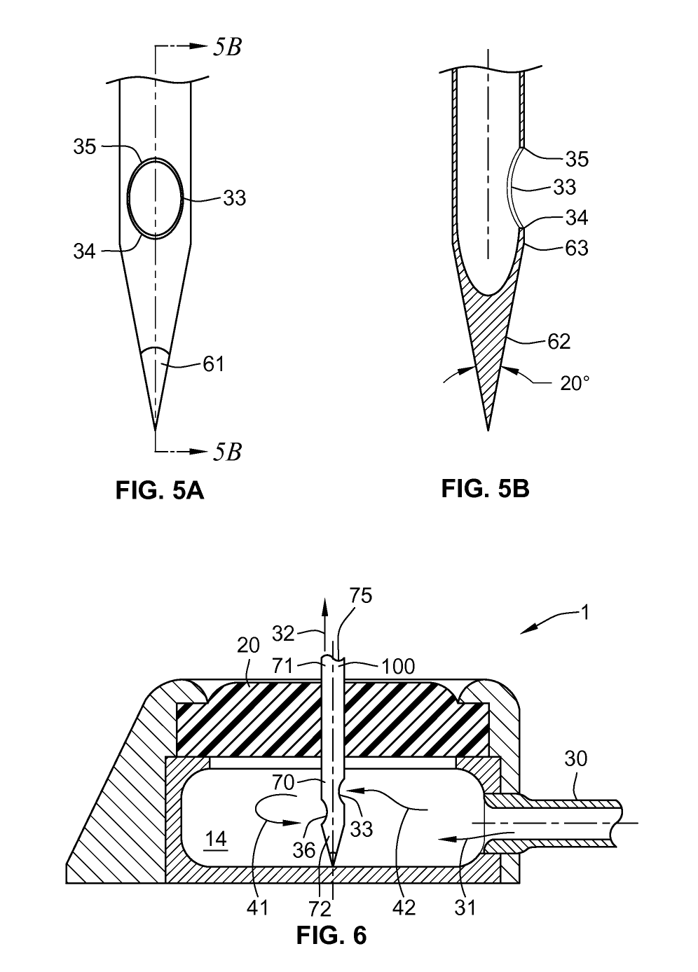

FIG. 4 illustrates a needle 100 with a single oval opening 33. FIG. 6 shows a needle 100 with two staggered oval openings 33, 36, each for collecting a fraction of the fluid from the cavity 14. Returning to FIG. 4, the needle is shown in greater detail in FIGS. 5A-5B and includes a pointed tip 62 with an end tip 61 of 0.06 inch in length in one preferred embodiment. The pointed tip 62 in another embodiment is a 20° cone. The inside portion of the cone shown in FIG. 5B includes a bottom resting place 63 shown as a semicircular surface to help stabilize the inner flow in the needle 100. What is contemplated is the use of a resting place 63 of such geometry to help with manufacturing while providing the greatest laminar flow within the main body of the needle 100.

The use of a vertical oval needle tip allows the creation of a greater opening surface than a regular or circular hole without weakening the body of the needle 100 at any portion of the needle along its vertical axis by not removing any metal in the radial orientation. FIG. 6 is another configuration where no portion of the needle 100 is weakened by placing two different openings along a single longitudinal radius. Two successive openings are staggered at different radial positions, shown to be at 180 degree or on opposite side of the needle. FIG. 8 shows a configuration where a grid of smaller holes 47 can be used and placed in a radial staggered configuration to draw in blood. In one preferred embodiment, the smaller holes 47 cannot be made to a size smaller than 5 to 10 times the total cross-section of 34 μm of the cells in the blood, or a size of 170 to 340 μm (0.0068 to 0.0136 in.). In yet another preferred embodiment, the circular opening diameter is 0.042 inch and is offset from the cone by 0.035 inch.

These needle configurations with multiple openings can be flow calibrated either by inserting the needle partly into the port plenum so only a portion of the openings is in contact with the blood flow, or by using a partial and movable cover.

For each of the embodiments shown, the edges of the different openings are rounded as shown with greater detail as 34 and 35 in FIG. 5A. What is also contemplated is the use of internal edges to direct the incoming flow in a selected direction to prevent the formation of vortices within the needle. What is also contemplated is the use of different walls or separations within the needle 100 to further direct the flow.

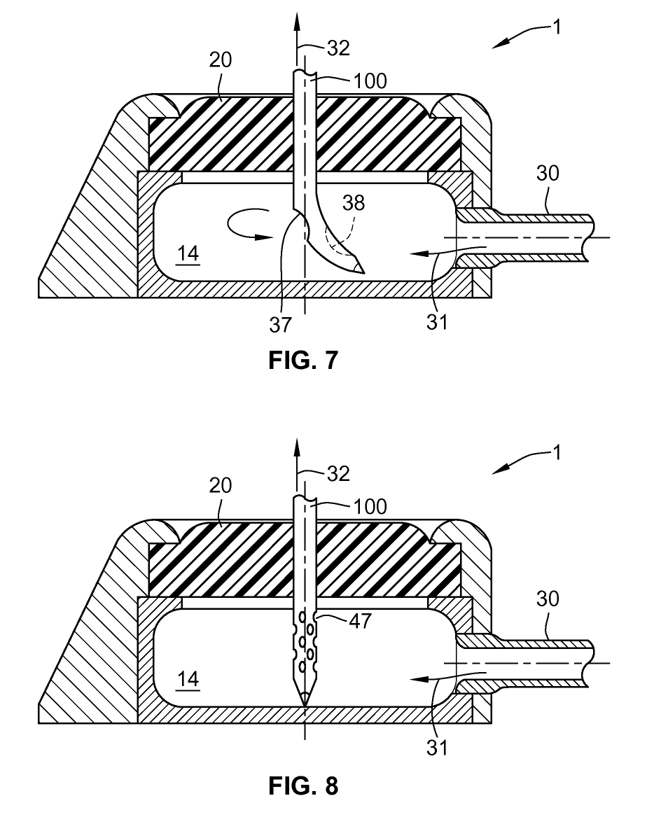

In one preferred embodiment, the internal diameter (d) of the needle 100 is taken to be 0.0525 to 0.0545 in. The external diameter of the needle 100 is taken to be 0.0645 to 0.0655 in. This corresponds to a minimum passage section of 0.0021 sq. in. (S=π(d/2)²). The surface of a circular opening of diameter 0.042 in. on the lateral wall of a needle is 0.0014 sq. in. (S=π(0.042/2)²) but for an oval opening made on a cylinder having a principal axis of 0.042 in. and a secondary axis of 1.5 times the principal axis 0.063 in., the surface can be approximated to 0.0021 sq. in. (S=πAB). The use of an opening with a passage area equal to the passage area of the needle 100 to prevent locally an increase in velocity in the blood is contemplated. As shown in FIG. 7, the use of a circular hole 37 placed on a bent needle or the use of two holes 37, 38 to regulate the flow of fluid through the needle is also contemplated.

What is also contemplated is the use of a permanent or a temporary coating placed on the needle to improve the flow inside of the needle, such as for example an anti-clouting coating like heparin, a bio-compatible coat like polished titanium oxide coatings, or even polymer coating such as, for example, Teflon or PTFE. In one embodiment, the coating is placed inside of the needle to facilitate the flow of blood. In another embodiment, the coating is place at the edges of the openings on the needle to reduce friction. In yet another embodiment (not shown), a sliding cover in the shape of a metallic shell can be retracted over a portion or the totality of the body of the needle. The placement of the cover allows for the control of the flow and the protection of the needle. In yet another embodiment, instead of a Huber needle, a regular needle with a cylindrical entry surface can be used in tandem with a pull out rod with pointed tip (not shown). In a first step of a method of use, the pointed rod is pushed passed the tip of the needle and enters the skin until the external perimeter of the needle contacts with the outer layer of the skin. The needle is then pushed in, and finally, the pull out rod is pulled out leaving the needle in place and allowing the flow of blood in the needle to start.

In yet another embodiment, as shown in FIG. 8, an intermediate portion of the needle can be manufactured of an array of small rounded strings of metal formed into a cylindrical mesh for allowing the passage of blood and welded to the end of the needle in the shape of a Huber tip. In yet another embodiment, the mesh is not angled and a Huber shape tip is connected to the mesh.

What is described is a needle 100 for a subcutaneous port 1 adapted to reduce the damage to the floating particles, such as blood cells a fluid at the inlet of the needle, the needle 100 having a needle shaft 70 with a bore 75 along a longitudinal axis of the needle shaft 70 with a proximal end 71 and a distal end 72 in opposition thereof as shown on FIG. 4, a pointed tip 62 at the distal end 72 with a pointed end tip 61 for the entry of at least a portion of the needle shaft shown as FIGS. 5A-B into a fluid reservoir 14 in the subcutaneous port 1. In addition, at least an inlet orifice or opening 33 along the needle shaft 70 between the proximal end 71 and the distal end 72 and in fluidic contact as shown by arrows 31, 32, with the fluid reservoir 14 and adjacent to the pointed tip 62. The inlet orifice 33 communicates with the bore 75 for the passage of the fluid from the fluid reservoir 14 through the inlet orifice 33 and through the bore 75 as shown by arrow 32. Further, the inlet orifice 33 has at least a rounded edge 34 or 35.

The inlet orifice may be of different shapes as shown including oval shape as shown on FIG. 4, and where oval shape has a long axe along the longitudinal axis of the shaft 70. The needle shaft 70 may have a thickness in the range of 0.001 to 0.003 inch. While some ranges and dimensions are given, one of ordinary skill in the art will recognize that any thickness is contemplated. In the embodiment shown as FIG. 7, the needle shaft 70 along the longitudinal axis is curved adjacent to the pointed tip 62. The plurality of orifices 47 or the grid of small holes are along the needle shaft 70 between the proximal end 71 and the distal end 72 and in fluidic contact with the fluid reservoir 14 and adjacent to the pointed tip 62, and where each of the plurality of inlet orifices as shown communicate with the bore 75 for the passage of fluid as shown by the arrows 31, 32 from the fluid reservoir 14 through the inlet orifice 33 and through the bore 75.

What is also contemplated is a method of protecting blood cells from damage during a medical treatment with a subcutaneous port 1, where blood is circulated through a needle 31, 32, the method having the steps of connecting (not shown) a needle 100 to a medical treatment device such as a hemodialysis machine for conducting a treatment using multiple circulation of blood through the needle 100, the needle 100 having a needle shaft 70 with a bore 75 along a longitudinal axis shown by the dashed line on FIGS. 4 to 6, and 8 of the needle shaft 70 and a proximal end 71 and a distal end 72 in opposition thereof, a pointed tip 62 at the distal end 72 with a pointed end tip 61, and at least an inlet orifice 33 along the needle shaft between the proximal end 71 and the distal end 72, and where the inlet orifice 33 has at least a rounded edge 34, 35 for the protection of blood cells. In a subsequent step, the plenum surface 20 as shown on FIG. 6 is punched for entry of at least a portion of the needle shaft 70 and the inlet orifice 33 into a fluid reservoir 14 in the subcutaneous port 1. The inlet orifice 33 is then placed in fluidic contact as shown by arrows 31, 41, 42, and ultimately 32 on FIG. 6 with blood in the fluid reservoir for the passage of the blood from the fluid reservoir 14 through the inlet orifice 33 and through the bore 75. Finally, the machine is then put on for the circulation of the blood so the flow of blood circulates around the rounded edge 33. In addition, openings are designed so the flow is not accelerated in the vicinity of the edges by having a plurality of openings in a single needle.

It is understood that the preceding is merely a detailed description of some examples and embodiments of the present invention and that numerous changes to the disclosed embodiments can be made in accordance with the disclosure made herein without departing from the spirit or scope of the invention. The preceding description, therefore, is not meant to limit the scope of the invention but to provide sufficient disclosure to one of ordinary skill in the art to practice the invention without undue burden.

What is claimed is:

1. A needle for a subcutaneous port adapted to reduce the damage to a biological or physiological fluid at the inlet of the needle, comprising:
a needle shaft with a bore along a longitudinal axis of the needle shaft and a proximal end and a distal end in opposition thereof;
a pointed tip at the distal end with a pointed end tip for the entry of at least a portion of the needle shaft into a biological or physiological fluid reservoir in the subcutaneous port; and
at least an inlet orifice along the needle shaft between the proximal end and the distal end and in fluidic contact with the biological or physiological fluid reservoir and adjacent to the pointed tip, wherein said inlet orifice communicates with the bore for the passage of the fluid from the biological or physiological fluid reservoir through the inlet orifice and through the bore, and wherein the inlet orifice has at least a rounded edge.

2. The needle of claim 1, wherein the inlet orifice is of oval shape.

3. The needle of claim 2, wherein the oval shape has a long axe along the longitudinal axis.

4. The needle of claim 3, wherein the pointed end tip at the pointed tip is a 20 degree cone.

5. The needle of claim 1, wherein the needle shaft has an exterior diameter in the range of 0.0645 to 0.0655 inch, and a bore of an internal diameter in the range of 0.0525 inch to 0.0545 inch.

6. The needle of claim 1, wherein the needle shaft has a thickness in the range of 0.001 to 0.003 inch.

7. The needle of claim 1, wherein the inlet orifice is a circular opening with a diameter in the range of 0.042 inch and is offset from the pointed end tip by approximately 0.035 inch.

8. The needle of claim 1, wherein at least two inlet orifices are along the needle shaft between the primal end and the distal end and in fluidic contact with the biological or physiological fluid reservoir and adjacent to the pointed tip, wherein each of the at least two inlet orifices communicate with the bore for the passage of the biological or physiological fluid from the biological or physiological fluid reservoir through the inlet orifice and through the bore, and wherein the inlet orifice has at least a rounded edge, and wherein each of the at least two inlet orifices are staggered at approximately 180 degree along the needle shaft.

9. The needle of claim 8, wherein the needle shaft wherein the longitudinal axis of is curved adjacent to the pointed tip.

10. The needle of claim 1, wherein a plurality of orifices are along the needle shaft between the proximal end and the distal end and in fluidic contact with the biological or physiological fluid reservoir and adjacent to the pointed tip, and wherein each of the plurality of inlet orifices communicate with the bore for the passage of fluid from the biological or physiological fluid reservoir through the inlet orifice and through the bore.

11. The needle of claim 10, wherein the plurality of orifices is a mesh.

12. The needle of claim 1, wherein a surface of the bore is coated with an anti-clotting.

13. The needle of claim 12, wherein the anti-clotting is heparin.

14. The needle of claim 1, wherein a surface of the bore includes a bio-compatible coating selected from a group consisting of polished titanium oxide, and polymer coating.

15. The needle of claim 14, wherein the polymer coating is selected from a group consisting of Teflon or PTFE.

16. The needle of claim 1, wherein an external surface of the needle shaft is coated with an anti-clotting.

17. A method of protecting blood cells from damage during a medical treatment with a subcutaneous port, where blood is circulated through a needle, the method comprising the steps of:
connecting a needle to a medical treatment device for conducting a treatment using multiple circulation of blood through the needle, the needle having a needle shaft having a bore along a longitudinal axis of the needle shaft and a proximal end and a distal end in opposition thereof, a pointed tip at the distal end with a pointed end tip, and at least an inlet orifice along the needle shaft between the proximal end and the distal end, and wherein the inlet orifice has at least a rounded edge for the protection of blood cells;
puncturing a plenum surface of a subcutaneous port for entry of at least a portion of the needle shaft and the inlet orifice into a fluid reservoir in the subcutaneous port;
placing the inlet orifice in fluidic contact with blood in the fluid reservoir for the passage of the blood from the fluid reservoir through the inlet orifice and through the bore; and circulating the blood so the flow of blood circulates around the rounded edge to protect the blood cells during the circulation to the medical treatment device.

18. The method of claim 17, wherein the medical treatment is hemodialysis.

19. The method of claim 17, wherein a diameter of the rounded edge is at least 5 to 10 times the total cross-section of blood cells in the blood.

20. The method of claim 17, wherein the inlet orifice is an oval has a long axe along the longitudinal axis

21. The needle of claim 1, wherein the biological or physiological fluid is blood.
